(19) United States
(12) Patent Application Publication
(10) Pub. No.: US 2019/0170752 A1
(43) Pub. Date: Jun. 6, 2019

(54) METHOD FOR PREDICTING EFFICACY OF IMMUNE CHECKPOINT INHIBITORS IN CANCER PATIENTS

(71) Applicant: BeiGene, Ltd., Camana Bay, Grand Cayman (KY)

(72) Inventors: Lusong Luo, Beijing (CN); Chengliang Dai, Beijing (CN); Zhijian Sun, Beijing (CN)

(73) Assignee: BeiGene, Ltd., Camana Bay, Grand Cayman (KY)

(21) Appl. No.: 16/323,410

(22) PCT Filed: Jul. 31, 2017
(86) PCT No.: PCT/CN2017/095144

§ 371 Date: Feb. 5, 2019

(30) Foreign Application Priority Data
Aug. 8, 2016 (CN) PCT/CN2016/093917

(51) Int. Cl. G01N 33/574; C12Q 1/02; C07K 16/28

(52) U.S. Cl. CPC G01N 33/574; C12Q 1/025; G01N 2500/10; G01N 2500/02; C07K 16/2818

(57) ABSTRACT

Disclosed is a method for predicting clinical response of a cancer patient to treatment with an immune checkpoint inhibitor, or a combination of two or more thereof, or with a combination of an immune checkpoint inhibitor and other antitumor drugs, by introducing bispecific T cell engager (BiTE) into the platform of lymphocytes containing T cells in vitro, which are cultured on feeder cells or in Matrigel®.

### CROSS REFERENCE TO RELATED APPLICATIONS

This application is a U.S. National Stage Application under 35 U.S.C. § 371 of International Application No. PCT/CN2017/095144, filed on Jul. 31, 2017, which claims the benefit of priority to International Application No. PCT/CN2016/093917 filed on Aug. 8, 2016, the entire contents of each of which are hereby incorporated by reference.

### FIELD OF THE INVENTION

Disclosed herein is a method for predicting clinical efficacy of an immune checkpoint inhibitor or a combination of two or more thereof or a combination of an immune checkpoint inhibitor and other antitumor drugs, or a method for screening an immune checkpoint inhibitor or a combination of two or more thereof or a combination of an immune checkpoint inhibitor and other antitumor drugs effective in the treatment of a cancer of interest and the new use of an BiTE antibody for predicting clinical efficacy of an immune checkpoint inhibitor or for drug screening. The methods disclosed herein are quick to carry out and in ex vivo or in vitro settings. Therefore, it is expected to have advantages in predicting clinical efficacy of an immune checkpoint inhibitor or a combination of two or more thereof or a combination of an immune checkpoint inhibitor and other antitumor drugs or in patient screening or drug screening.

### BACKGROUND OF THE INVENTION

Cellular immune responses are controlled by stimulatory and inhibitory pathways that act in concert under normal conditions. The ability of cancer cells to evade destruction by immune surveillance has been attributed to the aberrant production of signaling molecules, perhaps by tumor cells themselves or by cells in their vicinity, that trigger immune inhibitory pathways, that is, immune checkpoint pathways, thereby permitting tumors to persist and spread, e.g., Pardoll, Nature Reviews Cancer, 12: 252-264 (2012); Gelao et al., Toxins, 6: 914-933 (2014). This observation has provided the basis for promising new approaches to cancer treatment: if an immune inhibitory pathway turned on by a tumor could be reversed, then immune surveillance may be reactivated and tumors destroyed by a cytotoxic immune response against tumor cells. Initial tests of this approach have shown remarkable success and numerous clinical trials of immune checkpoint pathway inhibitors have been initiated, e.g., Pardoll (cited above); Gelao et al. (cited above). Targets of such inhibitors have included the CTLA-4 receptor, PD-1 receptor, and the PD-I ligand, PDL-1, as well as other inhibitory pathway components, e.g., Pardoll (cited above); Gelao et al. (cited above).

Redirecting the activity of T cells by bispecific antibodies against tumor cells, independently of their (T cells receptor) TCR specificity, is a promising approach to treat cancer [Riethmüller. Symmetry breaking: bispecific antibodies, the beginnings, and 50 years on. Cancer Immun. 2012; Frankel S R, Baeuerle P A. Targeting T cells to tumor cells using bispecific antibodies. Curr Opin Chem Biol. 2013; 17:385-92; Baeuerle P A, Reinhardt C. Bispecific T-cell engaging antibodies for cancer therapy. Cancer Res. 2009; 69:4941-4]. Antibodies that are bispecific for a surface target antigen on cancer cell and for T cell receptor (e.g., CD3) on T cells, is an alternative approach to engage T cell for cancer therapy.

Among hundreds of relatively unsuccessful bispecific antibody designs, a new class of bispecific antibodies called BiTE (for “bispecific T-cell engager”) antibodies have emerged as superior to other formats. These BiTE antibodies are relatively stable and easy to produce in mammalian cells and, are extremely potent even at low doses [Mack M, Riethmuller G, Kufer P A small bispecific antibody construct expressed as a functional single-chain molecule with high tumor cell cytotoxicity. Proc Natl Acad Sci USA 1995; 92: 7021-5; Loffler A, Kufer P Lutterbuse R, et al. A recombinant bispecific single-chain antibody, CD19 CD3, induces rapid and high lymphoma-directed cytotoxicity by unstimulated T lymphocytes. Blood. 2000; 95:2098-103; Kufer P, Mack M Gruber R, et al., Construction and biological activity of a recombinant bispecific single-chain antibody designed for therapy of minimal residual colorectal cancer. Cancer Immunol Immunother. 1997; 45:193-7. Mack M Gruber R, Schmidt S, et al., Biologic properties of a bispecific single-chain antibody directed against 17-1A (EpCAM) and CD3: tumor cell-dependent T cell stimulation and cytotoxic activity. J Immunol. 1997; 158:3965-70]. In general, BiTEs are polypeptides consisting of two scFvs in tandem with total molecular weights ranging from 55 to 60 kDa; they are typically produced as recombinant, non-glycosylated proteins secreted by higher eukaryotic cell lines. By definition, while their effector-binding arms bind specifically to T cell receptor (e.g., the epsilon subunit of CD3), their opposing target-binding arms can be directed against any number of epitopes, such as those differentially expressed on the surface of a tumor cell [Bryan D Choi, Mingqing Cai, Darell D Bigner, et al., Bispecific antibodies engage T cells for antitumor Immunotherapy. Expert Opin Biol Ther 2011; 11(7): 843-53].

BiTE proteins have the ability to initiate lytic synapses in the presence of tumor antigen, and experiments have demonstrated that robust T cell responses are possible even against tumor cells that do not express MHC class I. This triggers T cell activation, including release of cytotoxic molecules, cytokines and chemokines and induction of T cell proliferation [Riethmüller. Symmetry breaking: bispecific antibodies, the beginnings, and 50 years on. Cancer Immun. 2012; Frankel S R, Baeuerle P A. Targeting T cells to tumor cells using bispecific antibodies. Curr Opin Chem Biol. 2013; 17:385-92]. It was also demonstrated that they can reactivate TILs in the immunosuppressive microenvironment of the metastatic human tissue.

The first T cell bispecific antibody was described 30 years ago [Staerz U D, Kanagawa O, Bevan M J. Hybrid antibodies can target sites for attack by T cells. Nature. 1985; 314:628-31]. BiTE antibodies containing more than 10 different target antigens have so far been constructed, including CD19, EpCAM, Her2/neu, EGFR, CD66e (or CEA, CEACAM5), CD33, EphA2, and MCSP (or HMW-MAA) [Bargou R, Leo E, Zugmaier G, et al., Tumor regression in cancer patients by very low doses of a T cell-engaging antibody. Science. 2008; 321:974-7]. In lymphoma, one prominent, well-characterized BiTE is the recombinant anti-CD19×anti-CD3e construct, targeting the pan B-cell antigen, CD19 [Bargou R, Leo E, Zugmaier G, et al., Tumor regression in cancer patients by very low doses of a T cell-engaging antibody. Science. 2008; 321:974-7]. The first BiTE for systemic administration to cancer patients, blinatumomab (AMG103, Blincyto®), an anti-CD19×anti-CD3e BiTE, was recently approved by the U.S. FDA for the treatment of relapsed/refractory B cell acute lymphocytic leukemia (B-ALL) [Frankel S R, Baeuerle P A. Targeting T cells to tumor cells using bispecific antibodies. Curr Opin Chem Biol. 2013; 17:385-92]. The second BiTE construct tested in clinical trials is MT110 (AMG110), an anti-EpCAM×CD3 BiTE, which was designed to target EpCAM on a wide array of solid tumors in patients with lung, gastric, and colorectal cancers [Bryan D Choi, Mingqing Cai, Darell D Bigner, et al., Bispecific antibodies engage T cells for antitumor Immunotherapy. Expert Opin Biol Ther 2011; 11(7): 843-53].

It was demonstrated that the BiTE platform has great potential in the treatment of malignant diseases. Further investigations are needed to study the impact of regulatory T cells, the contribution of genuine tumor specific T-cell responses to clinical activity, possible mechanisms to evade BiTE-engaged T cells, and the broader utility of new BiTE antibodies for treatment of malignant diseases [P4944 Patrick A. Baeuerle and Carsten Reinhardt. Bispecific T-Cell Engaging Antibodies for Cancer Therapy. Cancer Res. 2009; 69: (12):4941-4].

Although many patients seem to benefit from the new treatments, results are not uniform for all patients; thus, there is a critical need for developing biomarkers that permit identification of patients that will benefit from a particular treatment.

The development of accurate experimental models of cancer is always a key challenge for both the investigation of basic cancer biology and the development of novel therapeutic modalities. Although established transformed cancer cell lines are easy to expand and experimentally manipulated, they frequently bear little resemblance to the tissues from which they were derived. Therefore, two-dimensional (2D) primary cell culture is becoming an important tool for providing predictions of drug activity, metabolism and toxicity in vitro.

Furthermore, to better represent the native architecture and cellular heterogeneity observed in the tumors, researchers have developed organotypic cancer models (organoids) via various 3D culture methods. Organoids are generally cultured in a 3D matrix that serves as a biomimetic scaffold, and the composition of this matrix can have significant effects on the cellular architecture and behavior of these cultures. The matrices routinely used for organoid cultures are Matrigel. This method provides not only a good environment for the primary tumor cells growth, but also a physiologically relevant and feasible means to study the mechanisms for anticancer agents.

It is well recognized that one of the key challenges in developing immune-oncology agents is to evaluate their activities as single agent or in combinations by predictive and robust preclinical models. Current in vitro/ex vivo studies typically do not replicate the full immunity cycle or have representative tumor microenvironment (TME). On the other hand, traditional xenograft or patient derived xenograft (PDX) models do not have representative TME for human tumors as typically immune-deficient mice are required for xenograft models. Syngeneic mouse models are currently the mainstay for evaluating Immuno-oncology Agents. However, these models have mouse TME, which could be different from human TME. In addition, many of the humanized antibodies do not bind to the mouse target proteins as well and oftentimes require surrogate mouse antibodies for evaluating human antibody drugs. This requirement has limited utilities of syngeneic mouse models in investigating and comparing immune-oncology agents. One of the potential solutions is to use Tumor Infiltrating Lymphocytes (TIL) and primary tumor cell cultures such as tumor organoids derived from same patient surgery tumor tissues to characterize the immuno-profile of tumor and TME. Additionally, such a system will enable us to test immune-oncology drugs as single agent or in combination and serve as a useful tool to direct clinical trial designs and to identify biomarkers. However, there are technical challenges in this approach that have limited the progresses in this field. It is known that complete T cell activation requires at least two signals. The first is delivered through the antigen-specific T cell receptor, whereas the second is generated by cognate interactions through adhesion molecules of T cells and antigen-presenting cells and/or by cytokines produced by antigen-presenting cells. One of the main hurdles for an ex vivo or in vitro system is that the activation of the immune system via tumor associated antigens (TAAs) is either missing or insufficient to induce strong antitumor immune responses. Therefore, it is very hard to detect changes of the anti-tumor immune response. Hence, it is critical to find ways to provide a first signal for T lymphocyte activation for ex vivo or in vitro cancer models.

The inventors of the present application, for the first time, utilize a BiTE as an engager to help T cell activation in the presence of tumor cells to predict clinical response of a cancer patient to treatment by an immune checkpoint inhibitor or a combination of two or more thereof or a combination of an immune checkpoint inhibitor and other antitumor drugs.

### SUMMARY OF THE INVENTION

Disclosed herein is a fast and ex vivo method for predicting clinical efficacy of an immune checkpoint inhibitor or a combination of two or more thereof or a combination of an immune checkpoint inhibitor and other antitumor drugs or a method for screening an immune checkpoint inhibitor or a combination of two or more thereof or a combination of an immune checkpoint inhibitor and other antitumor drugs effective in the treatment of a cancer of interest and the new use of an BiTE antibody for predicting clinical efficacy of an immune checkpoint inhibitor or for drug screening.

In a first aspect, disclosed herein is an ex vivo or in vitro method for predicting clinical response of cancer cells treated by an immune checkpoint inhibitor, or a combination of two or more thereof, a combination of an immune checkpoint inhibitor and other antitumor drugs, comprising
1) Contacting a co-culture system comprising the cancer cells and lymphocytes containing T cells with an BiTE antibody in the presence or absence of the immune checkpoint inhibitor or a combination of two or more thereof or a combination of an immune checkpoint inhibitor and other antitumor drugs; and
2) Determining whether the release of IFN-γ due to the activation of T cells by the BiTE antibody is increased in the presence of the immune checkpoint inhibitor or a combination of two or more thereof or a combination of an immune checkpoint inhibitor and other antitumor drugs as compared to the one absence thereof.

In an embodiment of the first aspect, the cancer cells are primary tumor cells from a cancer patient, or cancer cells of a cancer cell line, more preferably one corresponding to the cancer in the patient.

In an embodiment of the first aspect, the co-culture system is obtained by co-culturing the cancer cells with lymphocytes containing T cells.

In an embodiment of the first aspect, the BiTE antibody is added into the above co-culture system, and then divided into control groups and treatment groups.

In an embodiment of the first aspect, the immune checkpoint inhibitor or a combination of two or more thereof or a combination of an immune checkpoint inhibitor and other antitumor drugs is added into the above treatment groups.

In an embodiment of the first aspect, the increased release of IFN-γ is determined by measuring human IFN-γ from supernatant of the control groups and the treatment groups, to produce a cIFN-γ amount and a tIFN-γ amount, respectively, wherein a tIFN-γ amount higher than a cIFN-γ amount indicates that the patient is more likely to respond to the immune checkpoint inhibitor or a combination of two or more thereof or a combination of an immune checkpoint inhibitor and other antitumor drugs. Preferably, the increased release of IFN-γ is determined by the value of the tIFN-γ/cIFN-γ ratio, i.e., by dividing the tIFN-γ amount by the cIFN-γ amount, wherein a tIFN-γ/cIFN-γ ratio number higher than 1.0, 1.3, 1.5, 2.0, 2.5, 3.0, 3.5, 4, 4.5, 5, 6, 7, 8, 9 or 10 or more indicates that the patient is more likely to respond to the immune checkpoint inhibitor or a combination of two or more thereof or a combination of an immune checkpoint inhibitor and other antitumor drugs.

In a preferred embodiment of the first aspect, disclosed herein is an ex vivo or in vitro method for predicting clinical response of a cancer patient to treatment by an immune checkpoint inhibitor or a combination of two or more thereof or a combination of an immune checkpoint inhibitor and other antitumor drugs, comprising:
(a) co-culturing primary tumor cells from a cancer patient, or cancer cells of a cancer cell line, preferably one corresponding to the cancer in the patient, with lymphocytes containing T cells, to obtain a co-culture system;
(b) adding a BiTE into the co-culture system of (a), and dividing into control groups and treatment groups;
(c) adding an immune checkpoint inhibitor or a combination of two or more thereof or a combination of an immune checkpoint inhibitor and other antitumor drugs into the treatment groups of (b); and
(d) measuring human IFN-γ from supernatant of the control groups and the treatment groups, to produce a cIFN-γ amount and a tIFN-γ amount, respectively, wherein a tIFN-γ amount higher than a cIFN-γ amount indicates that the patient is more likely to respond to the immune checkpoint inhibitor or a combination of two or more thereof or a combination of an immune checkpoint inhibitor and other antitumor drugs.

In the method as discussed above, a tIFN-γ/cIFN-γ ratio is determined by dividing the tIFN-γ amount by the cIFN-γ amount, wherein a tIFN-γ/cIFN-γ ratio number higher than 1.0, 1.3, 1.5, 2.0, 2.5, 3.0, 3.5, 4, 4.5, 5, 6, 7, 8, 9 or 10 or more indicates that the patient is more likely to respond to the immune checkpoint inhibitor or a combination of two or more thereof or a combination of an immune checkpoint inhibitor and other antitumor drugs.

In a second aspect, disclosed herein is a method for screening a cancer patient who may benefit from treatment by an immune checkpoint inhibitor or a combination of two or more thereof or a combination of an immune checkpoint inhibitor and other antitumor drugs, comprising:
1) Contacting a co-culture system comprising primary tumor cells from the cancer patient and lymphocytes containing T cells with an BiTE antibody in the presence or absence of the immune checkpoint inhibitor or a combination of two or more thereof or a combination of an immune checkpoint inhibitor and other antitumor drugs; and
2) Determining whether the release of IFN-γ due to the activation of T cells by the BiTE antibody is increased in the presence of the immune checkpoint inhibitor or a combination of two or more thereof or a combination of an immune checkpoint inhibitor and other antitumor drugs as compared to the one absence thereof.

In an embodiment of the second aspect, disclosed herein is a method for screening a cancer patient who may benefit from treatment by an immune checkpoint inhibitor or a combination of two or more thereof or a combination of an immune checkpoint inhibitor and other antitumor drugs, comprising:
(a) co-culturing primary tumor cells from a cancer patient with lymphocytes containing T cells, to obtain a co-culture system;
(b) adding a BiTE into the co-culture system of (a), and dividing into control groups and treatment groups;
(c) adding an immune checkpoint inhibitor or a combination of two or more thereof or a combination of an immune checkpoint inhibitor and other antitumor drugs into the treatment groups of (b); and
(d) measuring human IFN-γ from supernatant of the control groups and the treatment groups, to produce a cIFN-γ amount and a tIFN-γ amount, respectively, wherein a tIFN-γ amount higher than a cIFN-γ amount indicates that the patient is more likely to benefit from the immune checkpoint inhibitor or a combination of two or more thereof or a combination of an immune checkpoint inhibitor and other antitumor drugs.

In another embodiment of the second aspect, disclosed herein is a method for screening cancer patients who will benefit from treatment by an immune checkpoint inhibitor or a combination of two or more thereof or a combination of an immune checkpoint inhibitor and other antitumor drugs, comprising:
(a) co-culturing primary tumor cells from a cancer patient with lymphocytes containing T cells, to obtain a co-culture system;
(b) adding a BiTE into the co-culture system of (a), and dividing into control groups and treatment groups;
(c) adding an immune checkpoint inhibitor or a combination of two or more thereof or a combination of an immune checkpoint inhibitor and other antitumor drugs into the treatment groups of (b);
(d) measuring human IFN-γ from supernatant of the control groups and the treatment groups, to produce a cIFN-γ amount and a tIFN-γ amount, respectively; and
(e) determining a tIFN-γ/cIFN-γ ratio by dividing the tIFN-γ amount by the cIFN-γ amount, wherein a tIFN-γ/cIFN-γ ratio number higher than 1.0, 1.3, 1.5, 2.0, 2.5, 3.0, 3.5, 4, 4.5, 5, 6, 7, 8, 9 or 10 or more indicates that the patient is more likely to benefit from the immune checkpoint inhibitor or a combination of two or more thereof or a combination of an immune checkpoint inhibitor and other antitumor drugs.

In a further aspect, disclosed herein is a method for treating cancer by an immune checkpoint inhibitor or a combination of two or more thereof or a combination of an immune checkpoint inhibitor and other antitumor drugs in a patient, wherein said patient has been screened by the method herein as a patient, who may benefit from treatment by said immune checkpoint inhibitor or said combination of two or more thereof or said combination of an immune checkpoint inhibitor and other antitumor drugs.

In a third aspect, disclosed herein is a kit for predicting clinical response of a cancer patient to treatment by an immune checkpoint inhibitor or a combination of two or more thereof or a combination of an immune checkpoint inhibitor and other antitumor drugs, as described above, comprising a composition comprising an active agent, which is effective for predicting whether a cancer patient will respond to immune checkpoint inhibitors or not, and the active agent in said composition comprises an agent which forms a cytolytic synapse between T cells and tumor cells bringing them into close proximity and the resultant activation of T cells releases cytokines such as IFN-γ and elicits a cytolytic response by driving a highly effective target cell killing machinery.

In a further aspect, disclosed herein is a kit for screening a cancer patient who will benefit from treatment by an immune checkpoint inhibitor or a combination of two or more thereof or a combination of an immune checkpoint inhibitor and other antitumor drugs, as described above.

The kits disclosed herein comprise at least one reagent which can specifically bind and thus detect human IFN-γ. For example, the reagent can be an anti-human IFN-γ antibody, e.g., Cat. No. 502402 from Biolegend. The reagent can also be an anti-human IFN-γ antibody conjugated to a label (e.g., biotin), e.g., Cat. No. 502504 from Biolegend. The reagent (e.g., an antibody) bound to human IFN-γ can be detected directly or indirectly by a number of methods well known in the art, e.g., ELISA.

In a fourth aspect, disclosed herein is a method for screening an immune checkpoint inhibitor, or a combination of two or more thereof, effective in the treatment of a cancer of interest, comprising:
1) Contacting a co-culture system comprising primary tumor cells from the cancer patient and lymphocytes containing T cells with an BiTE antibody in the presence or absence of the immune checkpoint inhibitor or a combination of two or more thereof or a combination of an immune checkpoint inhibitor and other antitumor drugs; and
2) Determining whether the release of IFN-γ due to the activation of T cells by the BiTE antibody is increased in the presence of the immune checkpoint inhibitor or a combination of two or more thereof or a combination of an immune checkpoint inhibitor and other antitumor drugs as compared to the one absence thereof.

In a further embodiment of the fourth aspect, disclosed herein is a method for screening an immune checkpoint inhibitor or a combination of two or more thereof or a combination of an immune checkpoint inhibitor and other antitumor drugs, effective in the treatment of a cancer of interest, comprising:
(a) co-culturing cancer cells, preferably cancer cells of a cancer cell line corresponding to the cancer of interest, or primary cancer cells from one or more cancer patients, with lymphocytes containing T cells, to obtain a co-culture system;
(b) adding a BiTE into the co-culture system of (a), and dividing into control groups and treatment groups;
(c) adding an immune checkpoint inhibitor or a combination of two or more thereof or a combination of an immune checkpoint inhibitor and other antitumor drugs into the treatment groups of (b); and
(d) measuring human IFN-γ from supernatant of the control groups and the treatment groups, to produce a cIFN-γ amount and a tIFN-γ amount, respectively, wherein a tIFN-γ amount higher than a cIFN-γ amount indicates that the immune checkpoint inhibitor or a combination of two or more thereof or a combination of an immune checkpoint inhibitor and other antitumor drugs is effective in the treatment of the cancer of interest.

In a further embodiment of the fourth aspect, disclosed herein is a method for screening an immune checkpoint inhibitor or a combination of two or more thereof or a combination of an immune checkpoint inhibitor and other antitumor drugs, effective in the treatment of a cancer of interest, comprising:
(a) co-culturing cancer cells, preferably cancer cells of a cancer cell line corresponding to the cancer of interest, or primary cancer cells from one or more cancer patients, with lymphocytes containing T cells, to obtain a co-culture system;
(b) adding a BiTE into the co-culture system of (a), and dividing into control groups and treatment groups;
(c) adding an immune checkpoint inhibitor or a combination of two or more thereof or a combination of an immune checkpoint inhibitor and other antitumor drugs into the treatment groups of (b);
(d) measuring human IFN-γ from supernatant of the control groups and the treatment groups, to produce a cIFN-γ amount and a tIFN-γ amount, respectively; and
(e) determining a tIFN-γ/cIFN-γ ratio by dividing the tIFN-γ amount by the cIFN-γ amount, wherein a tIFN-γ/cIFN-γ ratio number higher than 1.0, 1.3, 1.5, 2.0, 2.5, 3.0, 3.5, 4, 4.5, 5, 6, 7, 8, 9 or 10 or more indicates that the immune checkpoint inhibitor or a combination of two or more thereof or a combination of an immune checkpoint inhibitor and other antitumor drugs is effective in the treatment of the cancer of interest.

As disclosed herein, the above methods are performed in vitro (using cancer cells of a cancer cell line) or ex vivo or in vitro (using cancer cells from a cancer patient).

In a fifth aspect, disclosed herein is a BiTE molecule for use in a method for predicting clinical response of an immune checkpoint inhibitor or drug screening.

In an embodiment of the fifth aspect, disclosed herein is a BiTE molecule for use in a method for predicting clinical response of a cancer patient to treatment by an immune checkpoint inhibitor or a combination of two or more thereof or a combination of an immune checkpoint inhibitor and other antitumor drugs, wherein
1) the BiTE antibody is contacted with a co-culture system comprising primary tumor cells from the cancer patient and lymphocytes containing T cells in the presence or absence of the immune checkpoint inhibitor or a combination of two or more thereof or a combination of an immune checkpoint inhibitor and other antitumor drugs; and
2) the release of IFN-γ due to the activation of T cells by the BiTE antibody is measured to determine whether the release of IFN-γ is increased in the presence of the immune checkpoint inhibitor or a combination of two or more thereof or a combination of an immune checkpoint inhibitor and other antitumor drugs as compared to the one absence thereof.

In a further aspect, disclosed herein is a BiTE molecule for use in a method for screening an immune checkpoint inhibitor or a combination of two or more thereof or a combination of an immune checkpoint inhibitor and other antitumor drugs effective in the treatment of a cancer of interest, wherein
1) the BiTE antibody is contacted with a co-culture system comprising primary tumor cells from the cancer patient and lymphocytes containing T cells in the presence or absence of the immune checkpoint inhibitor or a combination of two or more thereof or a combination of an immune checkpoint inhibitor and other antitumor drugs; and
2) the release of IFN-γ due to the activation of T cells by the BiTE antibody is measured to determine whether the release of IFN-γ is increased in the presence of the immune checkpoint inhibitor or a combination of two or more thereof or a combination of an immune checkpoint inhibitor and other antitumor drugs as compared to the one absence thereof.

The inventors of this application have established a platform of patient tumor sample-derived TIL (Tumor Infiltrating Lymphocyte) or patients' PBMC (Peripheral Blood Mononuclear Cell) samples coupling with tumor cells including primary human tumor cells cultured in 2D or 3D (on feeder cells or in Matrigel®) as well as tumor cell lines. Instead of being used as a drug, the BiTE antibody has been introduced into this system which has enabled the inventors to evaluate immune-modulation of immune checkpoint inhibitor or a combination of two or more thereof or a combination of an immune checkpoint inhibitor and other antitumor drugs. In each of the above five aspects, the concentration of the BiTE antibody is in a range of 10 ng/ml to 1000 ng/ml; preferably a range of 10 ng/ml to 400 ng/ml; more preferably a range of 20 ng/ml to 200 ng/ml. As an example, the introduction of BiTE will allow concurrent measurement of human IFN-γ from supernatant of control groups (that is, control-IFN-γ, or cIFN-γ amount) and immune checkpoint inhibitors alone or/and combination treatment groups (that is, treatment-IFN-γ, or tIFN-γ amount), and determination of the tIFN-γ/cIFN-γ ratio (by dividing the tIFN-γ amount by the cIFN-γ amount, producing the tIFN-γ/cIFN-γ ratio number). The ratio number bigger than 1.0, 1.3, 1.5, 2.0, 2.5, 3.0, 3.5, 4, 4.5, 5, 6, 7, 8, 9 or 10 or more indicates that the patient with cancer will respond to the immune checkpoint inhibitors or/and the combinations.

### BRIEF DESCRIPTION OF THE DRAWINGS

FIG. 1 shows the level of IFN-γ produced from pre-activated PBMCs in primary tumor cells/PBMCs co-culture system treated with anti-PD-1 antibodies in EpCAM×CD3 BiTE platform. Wherein the primary tumor cells were derived from a tumor biopsy of a patient with adenocarcinoma of the esophagogastric junction, and were cultured on feeder cells and co-cultured with pre-activated human PBMCs, with different concentration of EpCAM×CD3 BiTE and with or without different anti-PD-1 antibodies, which are Mab-1 (1 μg/mL), nivolumab (1 μg/mL) or pembrolizumab (1 μg/mL).

FIG. 2 shows the level of the PD-1 expression on pre-activated PBMCs. The pre-activated PBMCs were derived from the PBMCs of a healthy donor, which were pre-activated by an anti-CD3 antibody (eBioscience, 16-0037-85). Three days later, the pre-activated PBMCs were collected and stained with PE anti-PD-1 antibody and run flow cytometry.

FIG. 3 shows the level of IFN-γ produced from PBMCs in primary tumor cells cultured in Matrigel® (namely, organoids)/PBMCs co-culture system treated with Mab-1, in the EpCAM×CD3 BiTE platform. Wherein the organoids were derived from a surgical tumor tissue from liver metastasis of a patient with colorectal cancer, and were co-cultured with non pre-activated human PBMCs, with different concentration of BiTE and with or without Mab-1 (1 μg/mL).

FIG. 4 shows the level of IFN-γ produced from PBMCs in primary human tumor cells cultured in Matrigel®/PBMCs co-culture system treated with Mab-1 or (Mab-1+Compound 1), in the EpCAM×CD3 BiTE platform. The primary human tumor cells were derived from a surgical tumor tissue of a patient with colon cancer, and were co-cultured with non pre-activated allogeneic human PBMCs, with different concentration of BiTE and with or without Mab-1 (1 μg/mL) and Mab-1+Compound 1 (1 μg/mL+300 nM).

### DETAILED DESCRIPTION OF THE INVENTION

### Abbreviations

Throughout the detailed description and examples disclosed herein, the following abbreviations will be used:

| | |
|---|---|
| BITE | Bispecific T Cell engager |
| BSA | Bovine Serum Albumin |
| Conc. | concentration |
| DPBS | Dulbecco's Phosphate Buffered Saline |
| ELISA | Enzyme Linked Immunosorbent Assay |
| HRP | Horseradish Peroxidase |
| IFN-γ | Interferon-γ |
| MMC | Mitomycin C |
| PBMCs | Peripheral Blood Mononuclear Cells |
| PD-1 | Programmed Death 1 |
| PBS | Phosphate Buffered Saline |
| PBST | PBS added with 0.5% Tween 20 |
| RPMI-1640 | Roswell Park Memorial Institute-1640 |
| TMB | 3,3′,5,5′-Tetramethylbenzidine |

Medium

In one embodiment of each of the above aspects, the cancer cells and lymphocytes containing T cells are co-cultured in a medium comprising:

1) F12/DMEM 1:1 medium 500 mL (Gibco®, 31765-035);

2) Fetal Bovine Serum 50 mL (Gibco®, 10099-141);

3) Insulin-Transferrin-Selenium (ITS-G) 0.5× (Gibco®, 41400045);

4) Penicillin-Streptomycin 1× (Gibco, 15140122);

5) HEPES 1× (Gibco®, 15630080);

6) Glutamine 2 mM (Gibco®, 25030081);

7) MEM Non-Essential Amino Acids Solution 1× (Gibco®, 11140050); and

8) Sodium pyruvate 1× (Gibco®, 11360070).

In an preferred embodiment, the medium is Medium BGB:

1) F12/DMEM 1:1 medium 500 mL (Gibco®, 31765-035);

### 2) Fetal Bovine Serum 50 ml (Gibco®, 10099-141);

3) B-27 supplement 1× (Gibco®, 17504-044);
4) B2 supplement 1× (Gibco®, 17502-048);

### 5) Insulin-Transferrin-Selenium (ITS-G) 0.5× (Gibco®, 41400045);

### 6) N-Acty-L-Cysteine 1.25 mM (Sigma®, V900429);

7) L-Ascorbic acid 25 ug/ml (Sigma®, V900134);
8) Folic acid 3.5 uM (Sigma®, V900422);

### 9) Putrescine 180 uM (Sigma®, V900377);

### 10) [Leu15]-Gastrin 10 nM (Sigma®, SCP0151);

### 11) Selenium 1.2 uM (Gibco®, 51300-044);

### 12) Glucose 25 mM (Sigma®, V900392);

### 13) Beta-ME 0.2 mM (Sigma®, M7522-100ML);

14) Recombinant Human HB-EGF 10 ng/ml (Peprotech, 100-47);
15) Recombinant Human R-Spondin-1 1 μg/ml (Peprotech, 120-38);
16) Recombinant Human Noggin 100 ng/ml (Peprotech, 120-10C);

### 17) HEPES 1× (Gibco®, 15630080);

### 18) Glutamine 2 mM (Gibco®, 25030081);

### 19) MEM Non-Essential Amino Acids Solution 1× (Gibco®, 11140050);

20) Sodium pyruvate 1× (Gibco®, 11360070);

### 21) Penicillin-Streptomycin 1× (Gibco, 15140122); and

22) ROCK inhibitor y-27632 10 M (Sigma®, Y0503).

EpCAM×CD3 BiTE amino sequence as follows (SEQ ID NO 1):

MGWSCIILFLVATATGVHSDIKLQQSGAELARPGASVKMSCKTSGYTFTR
YTMHWVKQRPGQGLEWIGYINPSRGYTNYNQKFKDKATLTTDKSSSTAYM
QLSSLTSEDSAVYYCARYYDDHYCLDYWGQGTTLTVSSGGGGSGGGGSGG
GGSDIQLTQSPAIMSASPGEKVTMTCRASSSVSYMNWYQQKSGTSPKRWI
YDTSKVASGVPYRFSGSGSGTSYSLTISSMEAEDAATYYCQQWSSNPLTF
GAGTKLELKSGGGGSELVMTQSPSSLTVTAGEKVTMSCKSSQSLLNSGNQ
KNYLTWYQQKPGQPPKLLIYWASTRESGVPDRFTGSGSGTDFTLTISSVQ
AEDLAVYYCQNDYSYPLTFGAGTKLEIKGGGGSGGGGSGGGGSEVQLLEQ
SGAELVRPGTSVKISCKASGYAFTNYWLGWVKQRPGHGLEWIGDIFPGSG
NIHYNEKFKGKATLTADKSSSTAYMQLSSLTFEDSAVYFCARLRNWDEPM
DYWGQGTTVTVSSHHHHHH

Mab-1 amino sequence as follows (SEQ ID NO 2):

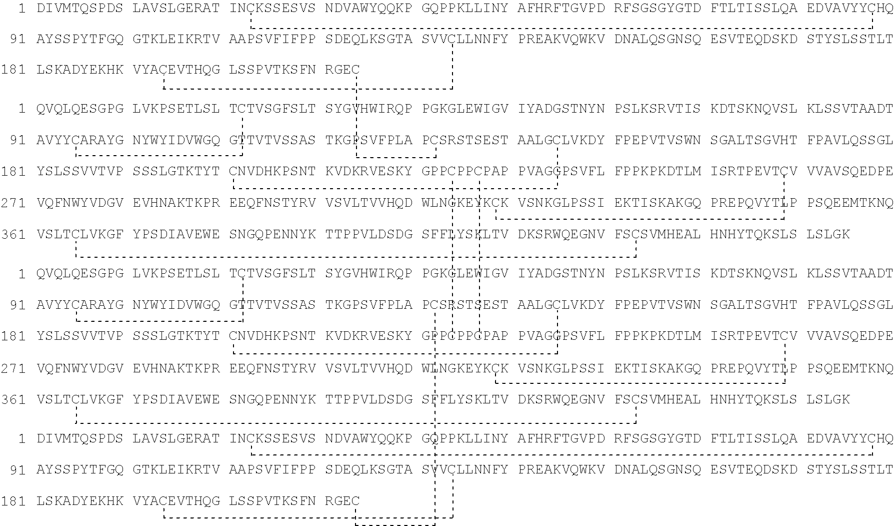

Exemplary Conservative Amino Acid Substitutions

| | Original | Conservative |
|---|---|---|
| Amino Acid Name | residue | substitution |
| Alanine | A or Ala | Gly; Ser |
| Arginine | R or Arg | Lys; His |
| Asparagine | N or Asn | Gln; His |
| Aspartic acid | D or Asp | Gln; Asn |
| Cysteine | C or Cys | Ser; Ala |
| Glutamine | Q or Gln | Asn |
| Glutamic acid | E or Glu | Asp; Gln |
| Glycine | G or Gly | Ala |
| Histidine | H or His | Asn; Gln |
| Isoleucine | I or Ile | Leu; Val |
| Leucine | L or Leu | Ile; val |
| Lysine | K or Lys | Arg; His |
| Methionine | M or Met | Leu; Ile; Tyr |
| Phenylalanine | F or Phe | Tyr; Met; Leu |
| Proline | P or Pro | Ala |
| Serine | S or Ser | Thr |
| Threonine | T or Thr | Ser |
| Tryptophan | W or Trp | Tyr; Phe |
| Tyrosine | Y or Tyr | Trp; Phe |
| Valine | V or Val | Ile; Leu |

### Definitions

Unless specifically defined elsewhere in this document, all the technical and scientific terms used herein have the meaning commonly understood by one of ordinary skill in the art to which this invention belongs.

As used herein, including the appended claims, the singular forms of words such as “a”, “an”, and “the”, include their corresponding plural references unless the context clearly dictates otherwise. Thus, for example, reference to “an immune checkpoint inhibitor” includes one or more of such different immune checkpoint inhibitors and reference to “the method” includes reference to equivalent steps and methods know to those of ordinary skill in the art that could be modified or substituted for the methods described herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

As used herein, the terms “patient” are used interchangeably and mean a mammal, including, but not limited to, a human or non-human mammal, such as a bovine, equine, canine, ovine, or feline. Preferably, the patient is a human.

As used herein, the terms “BiTE” or “Bispecific T-cell engager”, or “Bispecific Tcell engager”, also name BiTE antibody, refer to antibodies that are bispecific for a surface target antigen on cancer cell and for T cell receptor on T cells. The BiTE may be a bispecific single chain antibody construct containing at least two domains, wherein one of the domains specifically binds to antigens that are specific for human T cells including, but not limited to, CD3 (cluster of differentiation 3 T-cell co-receptor), CD4 (cluster of differentiation 4 on the surface of immune cells such as T helper cells, monocytes, macrophages, and dendritic cell), or CD8 (cluster of differentiation 8, a co-receptor for the T cell receptor, TCR) antigen (or CD4, CD8); and the second domain specifically binds to human tumor antigens including, but not limited to Epithelial cell adhesion molecule (EpCAM) antigen, Epidermal growth factor receptor (EGFR) antigen, Carcinoembryonic antigen (CEA) antigen, Epithelial tumor antigen (ETA) antigen, Alpha fetoprotein (AFP) antigen, Tyrosinase antigen, Melanoma-associated antigen (MAGE) antigen, abnormal products of ras, p53 antigen, Mucin 1, cell surface associated (MUC1) antigen, carcinoma antigen 125 (CA-125), human epidermal growth factor receptor 2 (Her2) antigen, B-lymphocyte antigen CD19 (CD19), and B-lymphocyte antigen CD20 (CD20). Numerous of BiTE are known, such as blinatumomab (AMG103, MT103/MEDI-538, Blincyto®), an anti-CD19×CD3 BiTE; MT110 (AMG110), an anti-EpCAM×CD3 BiTE; MT111 (MEDI-565), an anti-CEA×CD3 BiTE; A300E-BiTE, an anti-ADAM17×CD3 BiTE; MCSP×CD3 BiTE; EphA2×CD3 BiTE; and EGFR×CD3 BiTE. In some embodiments, the BiTE is a bispecific single chain antibody construct containing at least two domains, wherein one of the domains specifically binds to antigens selected from human CD3, CD4, or CD8 antigen, and the second domain specifically binds to human tumor antigens selected from EpCAM antigen, EGFR antigen, CEA antigen, Her2 antigen, CD19 or CD20. In some preferred embodiments, the BITE is anti-EpCAM×CD3 BITE, comprising an amino sequence of SEQ NO 1.

Immune checkpoints (checkpoint proteins) are molecules in the immune system that either turn up a signal (co-stimulatory molecules) or turn down a signal. And they also regulate T-cell activation or function. Many cancers protect themselves from the immune system by inhibiting the T cell signal. An “immune checkpoint inhibitor”, which totally or partially reduces, inhibits, interferes with or modulates one or more checkpoint proteins, has been increasingly considered as targets for cancer immunotherapies. Numerous checkpoint proteins are known, such as PD-1 (Programmed Death 1, CD-279) with its ligands PDL-1 and PDL-2; TIM-3 (T-cell Immunoglobulin domain and Mucin domain 3), and its ligand Gal-9; CTLA-4 (Cytotoxic T-Lymphocyte-Associated protein 4, CD152) and its ligands CD 80 and CD86; and A2AR (Adenosine A2A receptor); B7-H3 (CD276); B7-H4 (VTCN1); BTLA (B and T Lymphocyte Attenuator, CD272) and its ligand HVEM (Herpes virus Entry Mediator); IDO (Indoleamine 2,3-dioxygenase); LAG3 (Lymphocyte Activation Gene-3); VISTA (V-domain Ig suppressor of T-cell activation); KIR (Killer-cell Immunoglobulin-like Receptor). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The immune system has multiple inhibitory pathways that are critical for maintaining self-tolerance and modulating immune responses. In T-cells, the amplitude and quality of response is initiated through antigen recognition by the T-cell receptor and is regulated by immune checkpoint proteins that balance co-stimulatory and inhibitory signals.

PD-1 is an immune checkpoint protein, that limits the activity of T cells in peripheral tissues at the time of an inflammatory response to infection and to limit autoimmunity PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll, Nature Reviews Cancer, 12: 252-264, 2012). PD-1 blockade can be accomplished by a variety of mechanisms including antibodies that bind PD-1 or its ligands including: PDCD1, PD1, CD279 and SLEB2 for PD-1; PDCD1L1, PDL-1, B7H1, B7-4, CD274 and B7-H for PD-L1; and PDCDIL2, PDL2, B7-DC, Btdc and CD273 for PD-L2, PD-L1. Examples of PD-1 and PD-L1 blockers are described in U.S. Pat. Nos. 7,488,802; 7,943,743; 8,008,449; 8,168,757; 8,217,149, and WO03042402, WO2008156712, WO2010089411, WO2010036959, WO2011066342, WO2011159877, WO2011082400, WO2011161699, and WO2015035606. In some embodiments the PD-1 blockers include an antibody or a fragment antigen binding thereof, which specifically binds to PD-1. In certain other embodiments the PD-1 blockers include anti-PD-1 antibodies and similar binding proteins such as nivolumab (MDX 1106, BMS 936558, ONO 4538, Opdivo®) described in U.S. Pat. No. 8,008,449B2, a fully human IgG4 antibody that binds to and blocks the activation of PD-1 by its ligands PD-L1 and PD-L2; pembrolizumab (lambrolizumab, MK-3475 or SCH 900475, Keytruda®) disclosed in U.S. Pat. No. 8,168,757B2, a humanized monoclonal IgG4 antibody against PD-1; pidilizumab (CT-011), a humanized antibody that binds PD-1; AMP-224, a fusion protein of B7-DC; an antibody Fc portion; BMS-936559 (MDX-1105-01) for PD-L1 (B7-H1) blockade for PD-1 blockade.

Other immune checkpoint protein is CTLA-4, that down-regulates pathways of T-cell activation (Fong et al., Cancer Res. 69(2):609-615, 2009; Weber Cancer Immunol. Immunother, 58:823-830, 2009). Blockade of CTLA-4 has been shown to augment T-cell activation and proliferation. Inhibitors of CTLA-4 include anti-CTLA-4 antibodies. Anti-CTLA-4 antibodies bind to CTLA-4 and block the interaction of CTLA-4 with its ligands CD80/CD86 expressed on antigen presenting cells and thereby blocking the negative down regulation of the immune responses elicited by the interaction of these molecules. Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CDLA-4 antibody is tremelimumab (ticilimumab, CP-675,206). In one embodiment, the anti-CTLA-4 antibody is ipilimumab (MDX-010, MDX-101, Yervoy®), a fully human monoclonal IgG antibody that binds to CTLA-4. Ipilimumab is marketed under the name Yervoy™ and has been approved for the treatment of unresectable or metastatic melanoma. Other immune-checkpoint inhibitors include: LAG-3inhibitors, such as IMP321, a soluble Ig fusion protein (Brignone et al., 2007, J. Immunol. 179:4202-4211); B7 inhibitors, such as B7-H3 and B7-H4 inhibitors, e.g., anti-B7-H3 antibody MGA271 (Loo et al., 2012, Clin. Cancer Res. July 15 (18) 3834); TIM3 inhibitors; A2AR inhibitors; BTLA inhibitors; IDO inhibitors, e.g., INCB024360, an IDO1 inhibitor; VISTA inhibitors; or KIR inhibitors, such as lirilumab (INN), an antibody binding to KIR2DL1/2L3.

In some embodiments, the immune checkpoint inhibitor is an antibody or a fragment antigen binding thereof, or a chemical molecule drug. In some preferred embodiments, the immune checkpoint inhibitor is a chemical molecule drug, which inhibits one or more checkpoint proteins selected from human PD-1, TIM-3, A2AR, B7-H3 (CD276), B7-H4 (VTCN1), BTLA, CTLA-4, IDO, KIR, LAG3, or VISTA; or an antibody or a fragment antigen binding thereof, which specifically binds to one or more checkpoint proteins selected from human PD-1, TIM-3, A2AR, B7-H3 (CD276), B7-H4 (VTCN1), BTLA, CTLA-4, IDO, LAG3, or VISTA. In some more preferred embodiments, the immune checkpoint inhibitor is an antibody or a fragment antigen binding thereof, specifically binding to PD-1, which is selected from nivolumab, pembrolizumab, Mab-1 comprising an amino sequence of SEQ NO 2.

As used herein, the term “antibody” or “antibodies” includes reference to both glycosylated and non-glycosylated immunoglobulins of any isotype or subclass or to an antigen-binding region thereof that competes with the intact antibody for specific binding, unless otherwise specified, including monoclonal antibodies, bispecific antibodies, minibodies, domain antibodies, synthetic antibodies, antibody mimetics, chimeric antibodies, humanized antibodies, human antibodies, antibody fusions, antibody conjugates, single chain antibodies, antibody derivatives, antibody analogues and fragments thereof, respectively. Also included are immunological fragments of an antibody (e.g., a Fab, a Fab′, a F(ab′)2, or a scFv), irrespective of whether such antibodies are produced, in whole or in part, via immunization, through recombinant technology, by way of in vitro synthetic means, or otherwise. Thus, the term “antibody” is inclusive of those that are prepared, expressed, created or isolated by recombinant means, such as (a) antibodies isolated from an animal (e.g., a mouse) that is transgenic for human immunoglobulin genes or a hybridoma prepared therefrom, (b) antibodies isolated from a host cell transfected to express the antibody, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences. Such antibodies have variable and constant regions derived from germline immunoglobulin sequences of two distinct species of animals. In certain embodiments, however, such antibodies can be subjected to in vitro mutagenesis (or, when an animal transgenic for human immunoglobulin sequences is used, in vivo somatic mutagenesis) and thus the amino acid sequences of the VH and VL regions of the antibodies are sequences that, while derived from and related to the germline VH and VL sequences of a particular species (e.g., human), may not naturally exist within that species' antibody germline repertoire in vivo. Unless otherwise indicated, the term “antibody” includes, in addition to antibodies comprising two full-length heavy chains and two full-length light chains, derivatives, variants, fragments, and muteins thereof. In some instances “antibody” may include fewer chains such as antibodies naturally occurring in camelids which may comprise only heavy chains.

The term “monoclonal antibody” or “mAb” or “Mab” herein means a population of substantially homogeneous antibodies, i.e., the antibody molecules comprised in the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. Monoclonal antibodies (mAbs) may be obtained by methods known to those skilled in the art. See, for example Kohler et al (1975); U.S. Pat. No. 4,376,110; Ausubel et al (1987-1999); Harlow et al (1988); and Colligan et al (1993). The mAbs disclosed herein may be of any immunoglobulin class including IgG, IgM, IgD, IgE, IgA, and any subclass thereof. A hybridoma producing a mAb may be cultivated in vitro or in vivo. High titers of mAbs can be obtained in in vivo production where cells from the individual hybridomas are injected intraperitoneally into mice, such as pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as u, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (Vk/Vh) pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called “complementarity determining regions (CDRs)”, which are located within relatively conserved framework regions (FR). The CDRs are usually aligned by the framework regions, enabling binding to a specific epitope. In general, from N-terminal to C-terminal, both light and heavy chain variable domains comprise FR-1, CDR-1, FR-2, CDR-2, FR-3, CDR-3, and FR-4. The assignment of amino acids to each domain is, generally, in accordance with the definitions of Sequences of Proteins of Immunological Interest, Kabat, et al. National Institutes of Health, Bethesda, Md.; 5th ed.; NIH Publ. No. 91-3242 (1991); Kabat (1978) Adv. Prot. Chem. 32: 1-75; Kabat, et al., (1977) J. Biol. Chem. 252:6609-6616; Chothia, et al, (1987) J Mol. Biol. 196:901-917 or Chothia, et al, (1989) Nature 342:878-883.

The term “hypervariable region” means the amino acid residues of an antibody that are responsible for antigen-binding. The hypervariable region comprises amino acid residues from a “complementarity determining region” or “CDR” (i.e., CDR-L1, CDR-L2 and CDR-L3 in the light chain variable domain and CDR-H1, CDR-H2 and CDR-H3 in the heavy chain variable domain). See, Kabat et al. (1991) Sequences ofProteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, “antibody fragment” or “antigen binding fragment” means antigen binding fragments of antibodies, i.e., antibody fragments that retain the ability to bind specifically to the antigen bound by the full-length antibody, e.g., fragments that retain one or more CDR regions. Examples of antibody binding fragments include, but not limited to, Fab, Fab′, F(ab′)2, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. An antibody herein is said to bind specifically to a polypeptide comprising a given amino acid sequence, e.g. the amino acid sequence of a mature human PD-1 molecule, if it binds to polypeptides comprising that sequence but does not bind to proteins lacking that sequence.

The term “human antibody” herein means an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin sequences, respectively.

The term “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

As used herein, the terms “cancer” or “tumor” herein mean or describe the physiological condition in mammals that is typically characterized by unregulated cell growth. Examples of cancer include a solid tumor or a liquid tumor, but not limited to, gastric cancer, colon cancer, colorectal cancer, breast cancer, ovary cancer or a combination thereof.

As used herein, the terms “cancer cells” and “tumor cells” are interchangeably, comprising primary tumor cells from a cancer patient, or cancer cells of a cancer cell line.

The “combination” may be administered as a simultaneous, or separate or sequential regimen. When administered sequentially, the combination may be administered in two or more administrations. The combined administration includes co-administration, using separate formulation, and consecutive administration in either order, wherein preferably there is a time period while both (or all) active agents simultaneously exert their biological activities.

As used herein “other anti-tumor drug(s)” is a molecule that can treat, improve or delay cancer or tumor. In some embodiment, the other anti-tumor drug may have various modes of actions selected from immune therapies, targeted therapies, chemotherapies, vaccines, radiotherapies or combination thereof. As used herein, agents for chemotherapies include, but are not limited to, alkylating agents, anthracyclines, cytoskeletal disruptors (Taxanes), epothilones, histone deacetylase inhibitors, inhibitors of topoisomerase I, inhibitors of topoisomerase II, kinase inhibitors, nucleotide analogs and precursor analogs, peptide antibiotics, platinum-based agents, retinoids, and vinca alkaloids and derivatives. As used herein, agents for targeted therapies include, but are not limited to, EGFR inhibitors, ALK inhibitors, Bcl-2 inhibitors, PARP inhibitors, PI3K inhibitors, VEGFR-2 inhibitors, B-raf inhibitors, MEK inhibitors, CDK inhibitors, Hsp90 inhibitors, serine/threonine kinase inhibitors, and monoclonal antibodies.

In some preferred embodiments, the other anti-tumor drug is a chemical molecule drug, a proteasome inhibitor, a histone deacetylase inhibitor, a glucocorticoid, a steroid, a targeted therapy agent or combination thereof. In some more preferred embodiments, the other anti-tumor drug is an B-Raf inhibitor, such as 5-(((1R,1aS,6bR)-1-(6-(trifluoromethyl)-1H-benzo[d]imidazol-2-yl)-1a,6b-dihydro-1H-cyclopropa[b]benzofuran-5-yl)oxy)-3,4-dihydro-1,8-naphthyrdin-2(1H)-one or pharmaceutically acceptable salts thereof for the treatment of cancers with aberrations in the RAF-MEK-ERK MAPK pathway including B-Raf mutations, K-Ras/N-Ras mutations and NF1 mutations, which has potent and reversible inhibitory activities against RAF family kinases including A-Raf, B-Raf, C-Raf and B-RafV^{600E}, see WO 2013/097224A1. Particularly, the B-RAF inhibitor is 5-(((1R,1aS,6bR)-1-(6-(trifluoromethyl)-1H-benzo[d]imidazol-2-yl)-1a,6b-dihydro-1H-cyclopropa[b]benzofuran-5-yl)oxy)-3,4-dihydro-1,8-naphthyrdin-2(1H)-one Sesqui-Maleate (hereinafter referred as to Compound 1) disclosed in unpublished PCT application PCT/CN2016/079251.

As used herein “primary tumor cells” are cells isolated from a tumor tissue. In some embodiments, the primary tumor cells are isolated from a tumor biopsy. In some embodiments, the primary tumor cells are isolated from a tumor tissue sample of surgery. In some preferred embodiment the primary tumor cells are cultured on feeder cells, or in Matrigel®. In some more preferred embodiment the primary tumor cells were re-suspended with a medium comprising a substance listed as follows, before culturing.

1) F12/DMEM 1:1 medium 500 mL (Gibco®, 31765-035);

### 2) Fetal Bovine Serum 50 ml (Gibco®, 10099-141);

3) B-27 supplement 1× (Gibco®, 17504-044);
4) B2 supplement 1× (Gibco®, 17502-048);

### 5) Insulin-Transferrin-Selenium (ITS-G) 0.5× (Gibco®, 41400045);

### 6) N-Acty-L-Cysteine 1.25 mM (Sigma®, V900429);

7) L-Ascorbic acid 25 ug/ml (Sigma®, V900134);
8) Folic acid 3.5 uM (Sigma®, V900422);

### 9) Putrescine 180 uM (Sigma®, V900377);

### 10) [Leu15]-Gastrin 10 nM (Sigma®, SCP0151);

### 11) Selenium 1.2 uM (Gibco®, 51300-044);

### 12) Glucose 25 mM (Sigma®, V900392);

### 13) Beta-ME 0.2 mM (Sigma®, M7522-100ML);

14) Recombinant Human HB-EGF 10 ng/ml (Peprotech, 100-47);
15) Recombinant Human R-Spondin-1 1 μg/ml (Peprotech, 120-38);
16) Recombinant Human Noggin100 ng/ml (Peprotech, 120-10C);

### 17) HEPES 1× (Gibco®, 15630080);

### 18) Glutamine 2 mM (Gibco®, 25030081);

### 19) MEM Non-Essential Amino Acids Solution 1× (Gibco®, 11140050);

20) Sodium pyruvate 1× (Gibco®, 11360070); 21) Penicillin-Streptomycin 1× (Gibco, 15140122); and
22) ROCK inhibitor y-2763210 μM (Sigma®, Y0503).

T cells (or T lymphocytes) are a type of lymphocyte (a subtype of white blood cell) that play a central role in cell-mediated immunity. They can be distinguished from other lymphocytes, such as B cells and natural killer cells, by the presence of a T-cell receptor (TCR) on the cell surface. As used herein “lymphocytes containing T cells” are from different tissue such as peripheral blood, spleen, tumor, lymph node, ascites. “Peripheral blood mononuclear cells” or “PBMCs” are cells isolated from fresh human peripheral blood. “Tumor infiltrating lymphocytes” or “TILs” are cells isolated from tumor.

In some embodiments, the lymphocytes containing T cells are derived from peripheral blood, which provides PBMCs. In some preferred embodiments, the lymphocytes containing T cells are derived from allogeneic PBMCs, or autologous PBMCs. In particular, the allogeneic PBMCs are isolated from an allogeneic healthy human peripheral blood, and the autologous PBMCs can be isolated from autologous peripheral blood of the cancer patient. In some embodiments, the PBMCs are pre-activated by an antibody specifically binding to human CD3, CD4, or CD8 antigen, such as anti-CD3 antibody.

In some other embodiments, the lymphocytes containing T cells are derived from tissue selected from allogeneic TILs, autologous TILs, allogeneic lymphocytes from lymph node, autologous lymphocytes from lymph node, allogeneic spleen, autologous spleen, allogeneic ascites, or autologous ascites.

In some embodiments, the PBMCs can be replaced by purified CD3+ or CD8+ cells, wherein the CD3+ or CD8⁺ cells can be isolated from allogeneic and autologous human tumor tissue samples, lymph nodes, spleen or ascites, with ordinary methods in the art.

As used herein “control group” is a group compared to immune checkpoint inhibitors or a combination of two or more thereof, or a combination of an immune checkpoint inhibitor and anti-tumor drug treatment group. In some embodiments, the control group is without treatment by immune checkpoint inhibitor or a combination of two or more thereof, or a combination of an immune checkpoint inhibitor and anti-tumor drugs. In some further embodiments, the control group is treated with isotype control antibody, such as isotype control IgG.

In the present invention, the amounts of control-IFN-γ (cIFN-γ) and treatment-IFN-γ (tIFN-γ) are measured concurrently using a variety of technology platforms. They can be measured by immunodetection such as enzyme linked immunosorbent assay (ELISA), flow cytometry, and ELISPOT. And ELISA is a preferred method in immunodetection.

### Materials and Methods

PBMC Isolation and Activation

After signed the informed consent, healthy human donor's blood was collected and PBMCs were isolated with Ficoll (GE Healthcare, 17-1440-02) according to the manufacturer's protocol with small modification. Briefly, fresh blood was diluted with equivalent volume of 1×DPBS (Gibco®, 14190-144), added equivalent volume of Ficoll into a 50 mL sterile tube, and added diluted blood very carefully onto the surface of the Ficoll. Then centrifuged at 400 g for 40 minutes at room temperature. Carefully removed the upper layer and pipetted the lymphocyte layer to a clean sterile tube, added 3 volumes of 1×DPBS and centrifuge at 200 g for 10 minutes at room temperature. After discarded the supernatant, suspended cells with 10 mL 1×DPBS and washed 2 times more. Suspended cells with 5 mL RPMI-1640 (Gibco®, 22400-089) complete medium (added with 10% fetal bovine serum (Gibco®, 10099-141)).

For the activation of PBMCs, 5 mL 1×DPBS with 1 μg/mL anti-CD3 antibody (eBioscience, 16-0037-85) was added to a T25 plate (Corning, 430168), incubate at 4° C. overnight. Liquid in the T25 plate was discarded and the plate was washed with 5 mL 1×DPBS for 2 times, discarded 1×DPBS and added fresh isolated 6-8×10⁶ PBMCs suspended in 5 mL RPMI-1640 complete medium. Put the plate at 37° C., 5% CO₂ cell incubator. 2 to 3 days later, cells was collected and counted amount.

Primary Human Tumor Cell Isolation

After a patient with cancer signed the informed consent, tumor biopsy or surgical tumor tissue sample from patient was collected. At the day of tumor collection, tumor was sterilely minced to small pieces at a biosafety cabinet. Then tumor pieces was suspended with 10 mL digestion buffer, digested at 37° C. and 200 rpm for 0.5-1 hour. Undigested tumor pieces were removed by filtering with a 70 μm strainer. Liquid with tumor cells passed the strainer was centrifuged at 300 g for 5 minutes at room temperature. Discarded the supernatant and suspended cells with 10 mL 1×DPBS and washed 2 times more. The cells are re-suspended with Medium BGB.

Flow Cytometry Analysis

Activated PBMCs were collected and washed with 1×DPBS. 3×10⁵ cells were stained with a fluorescent probe conjugated anti-PD-1 antibody (eBioscience, 12-2799-42). The PD-1 expressed on PBMCs was analyzed by flow cytometry.

Culturing Primary Human Tumor Cells on Feeder Cells

C3H10T1/2 cell (ATCC, ATCC® CCL-226™) is a mouse embryo cell line and used as feeder cell. When the confluence of C3H10T1/2 cell was about 90%, cells were treated with 10 μg/mL mitomycin C (MCC, Sigma®, M0503) at 37° C. for 2 hours. Then discarded the medium with MMC and washed with 1×DPBS for 3 times. Cells were collected and stored in liquid-nitrogen. 4 hours before isolated primary tumor cells were seeded, MMC treated C3H10T1/2 cells were seeded into a 6-well plate. Two days after tumor cells were seeded, changed medium to remove unattached cells. Medium was changed according to the medium color. When the confluence was about 70-90%, primary tumor cells were trypsinized and subcultured to 96-well plate which was pre-seeded with MMC treated C3H10T1/2. Culture primary tumor cells to the confluence sufficient to co-culture with PBMCs.

Culturing Primary Human Tumor Cells in Matrigel®

Isolated primary tumor cells suspended in medium were mixed with 2% Matrigel® (BD, 356234) and then seeded in a 48-well plate. Medium was changed according to the medium color. When cells should be subcultured, organoids in Matrigel® were collected and pipetted to dispense the Matrigel® to small pieces and subcultured to a 96-well plate to co-cultrue with PBMCs.

ELISA for Human IFN-γ

Coated a 96-well high binding plate (Corning, 9018) with purified anti-human IFN-γ (Biolegend, 502402) at 37° C. for 2 hours or overnight, after washed the plate with 1×PBST (PBS added with 0.5% Tween 20 (Sigma®, P1379)) for 3 times, plate was blocked with 3% BSA (ChemCruz®, sc-2323) resolved in 1×PBST. Then supernatant of the co-culture system was incubated at room temperature for 2 hours or overnight. After washed the plate with 1×PBST for 3 times, plate was incubated with biotin conjugated anti-human IFN-γ (Biolegend, 502504) at room temperature for 1 hour. After washed the plate with 1×PBST for 3 times, plate was incubated with HRP conjugated streptavidin (Themo Scientific, 21130) at room temperature for 30 minutes. After washed the plate with 1×PBST for 5 times, 100 μL 1×TMB (ebioscience, 00-4201-56) was added to each well of the plate, 5 to 15 minutes later, each well was added with 50 μL of 2 mM H₂SO₄ to stop the reaction. Read absorbance at 450 nm.

### EXAMPLES

The present invention is further exemplified, but not limited, by the following examples that illustrate the invention. In the examples of the present invention, the techniques or methods, unless expressly stated otherwise, are conventional techniques or methods in the art.

### Example 1

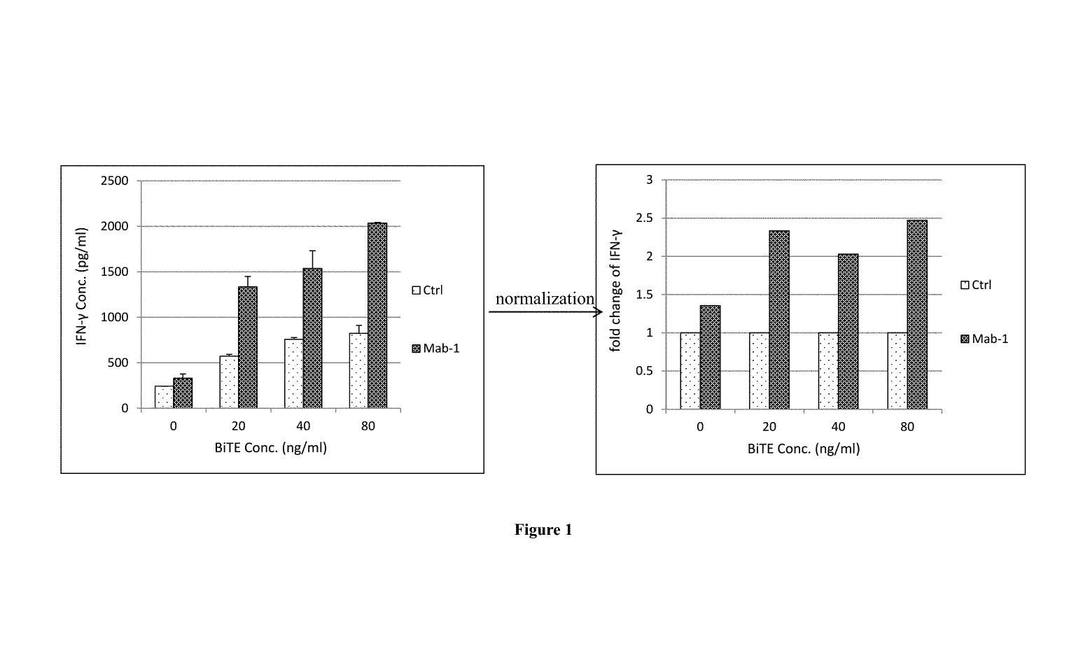

Primary tumor cells from a patient with adenocarcinoma of the esophagogastric junction cultured on feeder cells were co-cultured with pre-activated allogeneic human PBMCs, with different concentration of EpCAM×CD3 BiTE and with or without different anti-PD-1 antibody drugs (0.1 μg/mL), which are Mab-1 and cultured for 24 hours. Human IFN-γ in the supernatant of the co-culture system was measured and as the readout (See FIG. 1). The IFN-γ concentration of each BiTE concentration group was normalized to fold change by antibody treatment group concentration dividing by control group concentration.

At the group without BiTE, the fold changes of anti-PD-1 antibody treatment groups were all smaller than 1.3, which can be explained by the fact that PBMCs and tumor cells only physically contacted and there was no or litter immune synapse formation between PBMCs and tumor cells, PD-1 signal wasn't activated, little, if any. While at the groups with BiTE, the fold changes of anti-PD-1 antibody treatment groups were all bigger than 1.3, which indicated that the primary tumor cells responded to anti-PD-1 antibody treatment and predicted that there is a substantial possibility that this cancer patient will respond to the tested anti-PD-1 antibodies.

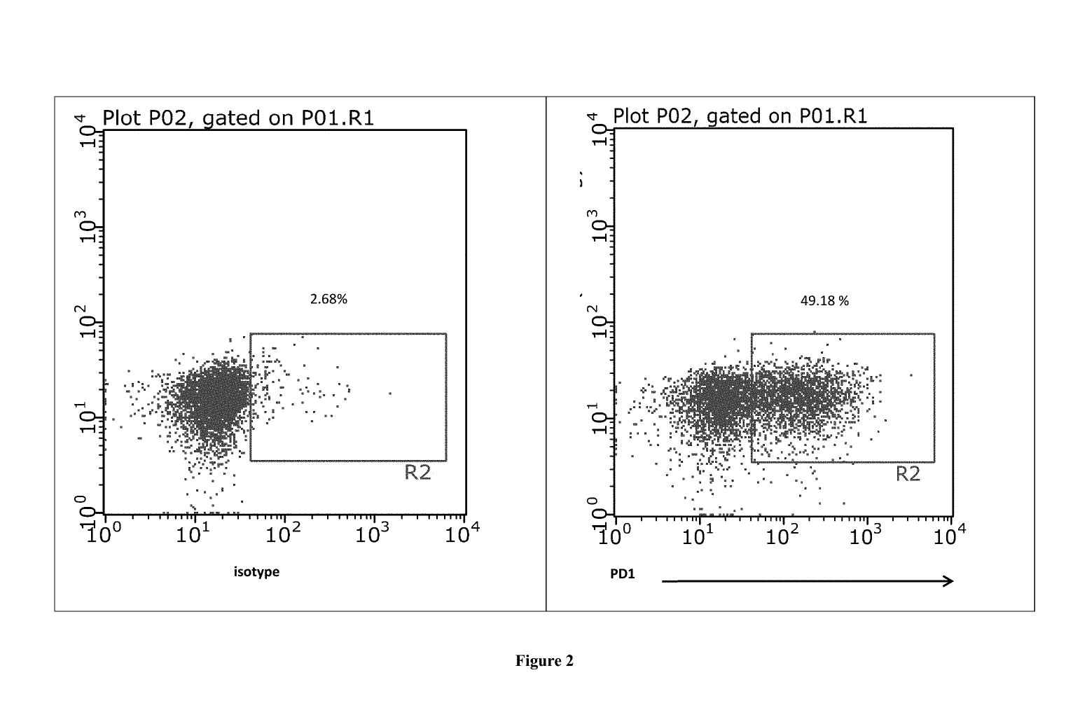

On the other hand, PD-1 expression on pre-activated PBMCs was measured. According to the responsive effect to anti-PD-1 antibodies, PD-1 expression on pre-activated PBMCs was significantly increased after pre-activated (See FIG. 2). So, the primary tumor cells cultured on feeder cells responded to those different anti-PD-1 antibodies in test.

### Example 2

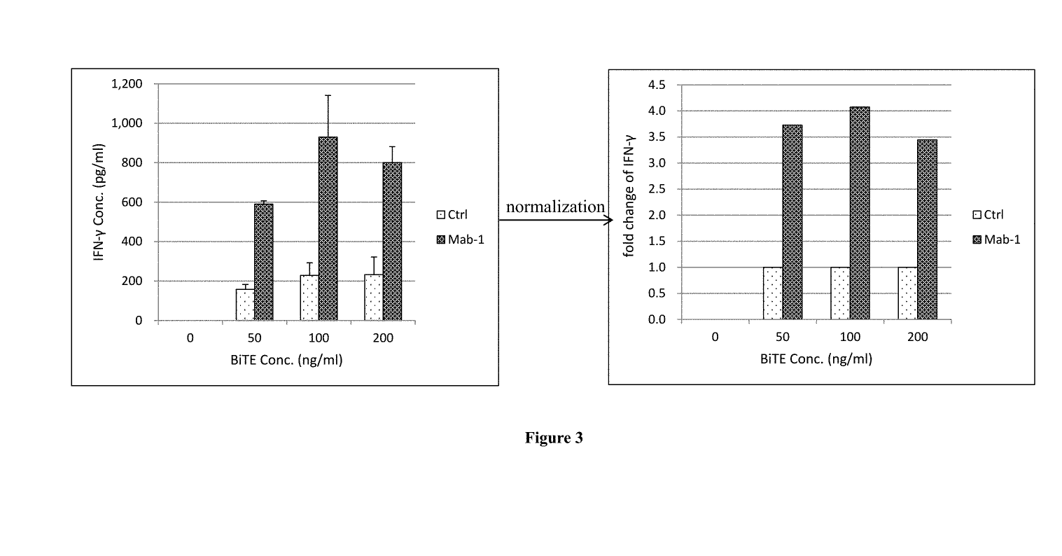

Primary tumor cells from a patient with colorectal cancer cultured in Matrigel® were co-cultured with non pre-activated allogeneic human PBMCs, with different concentration of EpCAM×CD3 BiTE and with or without 1 μg/mL of Mab-1 and cultured for 72 hours. Human IFN-γ in the supernatant of the co-culture system was measured and as the readout (See FIG. 3). The IFN-γ concentration of each BiTE concentration group was normalized to fold change by antibody treatment group concentration dividing by control group concentration.

At the groups without BiTE, Human IFN-γ can't be detected by ELISA. While at the groups with BiTE, the fold changes of Mab-1 treatment groups were all bigger than 1.3 and almost 3.5 times of control groups, which indicated that the primary tumor cells responded to Mab-1 treatment and predicted that there is a substantial possibility that this cancer patient will respond to the tested anti-PD-1 antibody. So, the primary tumor cells cultured in Matrigel® responded to anti-PD-1 antibody in test.

### Example 3

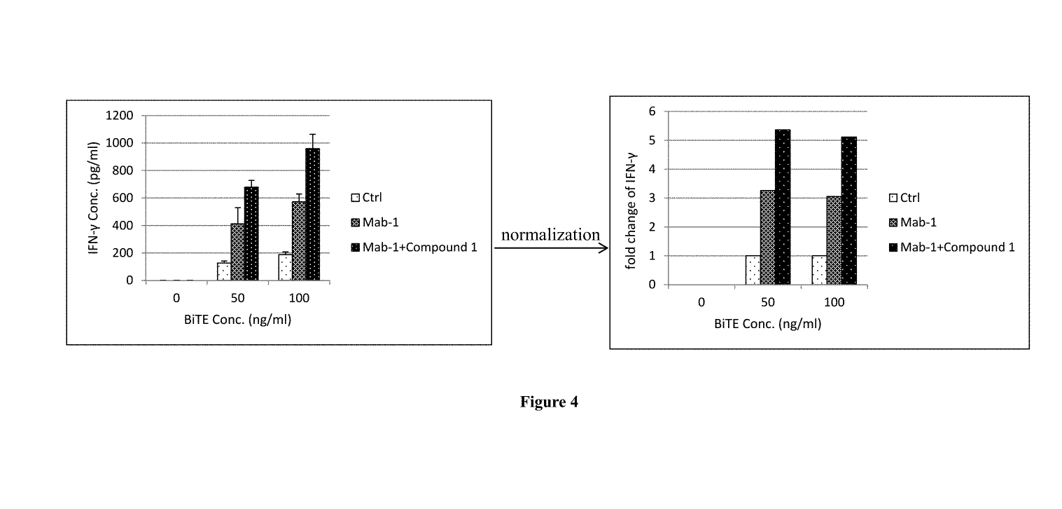

Primary tumor cells from a patient with colon cancer cultured in Matrigel® were co-cultured with non pre-activated allogeneic human PBMCs, with different concentration of EpCAM×CD3 BiTE and with or without an anti-PD-1 antibody named Mab-1 (1 μg/mL), and the combination of Mab-1 (1 μg/mL) and 5-(((1R,1aS,6bR)-1-(6-(trifluoromethyl)-1H-benzo[d]imidazol-2-yl)-1a,6b-dihydro-H-cyclopropa[b]benzofuran-5-yl)oxy)-3,4-dihydro-1,8-naphthyrdin-2(1H)-one Sesqui-Maleate (Compound 1, 300 nM) (named as Mab-1+Compound 1, for short), and cultured for 72 hours. Human IFN-γ in the supernatant of the co-culture system was measured and as the readout (See FIG. 4). The IFN-γ concentration of each BiTE concentration group was normalized to fold change by antibody treatment group concentration dividing by control group concentration. Primary tumor cells cultured in Matrigel® responded to anti-PD-1 antibody.

At the groups without BiTE, Human IFN-γ can't be detected by ELISA. While at the groups with BiTE, the fold changes of anti-PD-1 antibody and the combination treatment groups were all bigger than 1.3, the fold changes of combination was bigger than anti-PD-1 antibody alone treatment groups, which indicated that the primary tumor cells not only responded to anti-PD-1 antibody alone, but also there was significant synergistic effect of anti-PD-1 antibody and Compound 1 on primary tumor cells cultured in Matrigel®. These predicted that there is a possibility that this cancer patient will not only respond to the tested anti-PD-1 antibody alone but also the effect of its combination with Compound 1 will be better than anti-PD-1 antibody alone treatment. So, there was significant synergistic effect of anti-PD-1 antibody and Compound 1 on primary tumor cells cultured in Matrigel®.

1. An ex vivo or in vitro method for predicting clinical response of cancer cells treated by an immune checkpoint inhibitor or a combination of two or more thereof or a combination of an immune checkpoint inhibitor and other antitumor drugs, comprising
1) Contacting a co-culture system comprising the cancer cells and lymphocytes containing T cells with an bispecific T-cell engage antibody in the presence or absence of the immune checkpoint inhibitor or a combination of two or more thereof or a combination of an immune checkpoint inhibitor and other antitumor drugs; and
2) Determining whether the release of IFN-γ due to the activation of T cells by the bispecific T-cell engage antibody is increased in the presence of the immune checkpoint inhibitor or a combination of two or more thereof or a combination of an immune checkpoint inhibitor and other antitumor drugs as compared to the one absence thereof.

2. The method of claim 1, wherein the cancer cells are primary tumor cells from a cancer patient, or cancer cells of a cancer cell line.

3. The method of claim 1, wherein the co-culture system is obtained by co-culturing the cancer cells with lymphocytes containing T cells.

4. The method of claim 1, wherein the increased release of IFN-γ is determined by measuring human IFN-γ from supernatant in the absence or presence of the immune checkpoint inhibitor or a combination of two or more thereof or a combination of an immune checkpoint inhibitor and other antitumor drugs, to produce a cIFN-γ amount and a tIFN-γ amount, respectively, wherein a tIFN-γ amount higher than a cIFN-γ amount indicates that the patient is more likely to respond to the immune checkpoint inhibitor or a combination of two or more thereof or a combination of an immune checkpoint inhibitor and other antitumor drugs.

5. The method of claim 4 wherein the increased release of IFN-γ is determined by the value of the tIFN-γ/cIFN-γ ratio, i.e., by dividing the tIFN-γ amount by the cIFN-γ amount, wherein a tIFN-γ/cIFN-γ ratio number higher than 1.0, 1.3, 1.5, 2.0, 2.5, 3.0, 3.5, 4, 4.5, 5, 6, 7, 8, 9 or 10 or more indicates that the patient is more likely to respond to the immune checkpoint inhibitor or a combination of two or more thereof or a combination of an immune checkpoint inhibitor and other antitumor drugs.

6. The method of claim 1, for predicting clinical response of a cancer patient to treatment by an immune checkpoint inhibitor or a combination of two or more thereof or a combination of an immune checkpoint inhibitor and other antitumor drugs, comprising:
(a) co-culturing primary tumor cells from a cancer patient, or cancer cells of a cancer cell line, preferably one corresponding to the cancer in the patient, with lymphocytes containing T cells, to obtain a co-culture system;
(b) adding a bispecific T-cell engage into the co-culture system of (a), and dividing into control groups and treatment groups;
(c) adding an immune checkpoint inhibitor or a combination of two or more thereof or a combination of an immune checkpoint inhibitor and other antitumor drugs into the treatment groups of (b); and
(d) measuring human IFN-γ from supernatant of the control groups and the treatment groups, to produce a cIFN-γ amount and a tIFN-γ amount, respectively, wherein a tIFN-γ amount higher than a cIFN-γ amount indicates that the patient is more likely to respond to the immune checkpoint inhibitor or a combination of two or more thereof or a combination of an immune checkpoint inhibitor and other antitumor drugs.

7. A method for screening a cancer patient who may benefit from treatment by an immune checkpoint inhibitor or a combination of two or more thereof or a combination of an immune checkpoint inhibitor and other antitumor drugs, comprising:
1) Contacting a co-culture system comprising primary tumor cells from the cancer patient and lymphocytes containing T cells with an bispecific T-cell engage antibody in the presence or absence of the immune checkpoint inhibitor or a combination of two or more thereof or a combination of an immune checkpoint inhibitor and other antitumor drugs; and
2) Determining whether the release of IFN-γ due to the activation of T cells by the bispecific T-cell engage antibody is increased in the presence of the i immune checkpoint inhibitor or a combination of two or more thereof or a combination of an immune checkpoint inhibitor and other antitumor drugs as compared to the one absence thereof.

8. The method of claim 7, comprising:
(a) co-culturing primary tumor cells from a cancer patient with lymphocytes containing T cells, to obtain a co-culture system;
(b) adding a bispecific T-cell engage into the co-culture system of (a), and dividing into control groups and treatment groups;
(c) adding an immune checkpoint inhibitor or a combination of two or more thereof or a combination of an immune checkpoint inhibitor and other antitumor drugs into the treatment groups of (b); and
(d) measuring human IFN-γ from supernatant of the control groups and the treatment groups, to produce a cIFN-γ amount and a tIFN-γ amount, respectively, wherein a tIFN-γ amount higher than a cIFN-γ amount indicates that the patient is more likely to benefit from the immune checkpoint inhibitor or a combination of two or more thereof or a combination of an immune checkpoint inhibitor and other antitumor drugs.

9. The method of claim 8, wherein a tIFN-γ/cIFN-γ ratio is determined by dividing the tIFN-γ amount by the cIFN-γ amount, wherein a tIFN-γ/cIFN-γ ratio number higher than 1.0, 1.3, 1.5, 2.0, 2.5, 3.0, 3.5, 4, 4.5, 5, 6, 7, 8, 9 or 10 or more indicates that the patient is more likely to benefit from the immune checkpoint inhibitor or a combination of two or more thereof or a combination of an immune checkpoint inhibitor and other antitumor drugs.

10. A method for treating cancer by an immune checkpoint inhibitor or a combination of two or more thereof or a combination of an immune checkpoint inhibitor and other antitumor drugs in a patient, wherein said patient has been screened by the method according to any one of claims 7-9 as a patient, who may benefit from treatment by said immune checkpoint inhibitor or said combination of two or more thereof or said combination of an immune checkpoint inhibitor and other antitumor drugs.

11. A method for screening an immune checkpoint inhibitor or a combination of two or more thereof or a combination of an immune checkpoint inhibitor and other antitumor drugs, effective in the treatment of a cancer of interest, comprising:
1) Contacting a co-culture system comprising primary tumor cells from the cancer patient and lymphocytes containing T cells with an bispecific T-cell engage antibody in the presence or absence of the immune checkpoint inhibitor or a combination of two or more thereof or a combination of an immune checkpoint inhibitor and other antitumor drugs; and
2) Determining whether the release of IFN-γ due to the activation of T cells by the bispecific T-cell engage antibody is increased in the presence of the immune checkpoint inhibitor or a combination of two or more thereof or a combination of an immune checkpoint inhibitor and other antitumor drugs as compared to the one absence thereof.

12. The method of claim 11, comprising:
(a) co-culturing cancer cells, preferably cancer cells of a cancer cell line corresponding to the cancer of interest, or primary cancer cells from one or more cancer patients, with lymphocytes containing T cells, to obtain a co-culture system;
(b) adding a bispecific T-cell engage into the co-culture system of (a), and dividing into control groups and treatment groups;
(c) adding an immune checkpoint inhibitor or a combination of two or more thereof or a combination of an immune checkpoint inhibitor and other antitumor drugs into the treatment groups of (b); and
(d) measuring human IFN-γ from supernatant of the control groups and the treatment groups, to produce a cIFN-γ amount and a tIFN-γ amount, respectively, wherein a tIFN-γ amount higher than a cIFN-γ amount indicates that the immune checkpoint inhibitor or a combination of two or more thereof or a combination of an immune checkpoint inhibitor and other antitumor drugs is effective in the treatment of the cancer of interest.

13. The method of claim 12, wherein a tIFN-γ/cIFN-γ ratio is determined by dividing the tIFN-γ amount by the cIFN-γ amount, wherein a tIFN-γ/cIFN-γ ratio number higher than 1.0, 1.3, 1.5, 2.0, 2.5, 3.0, 3.5, 4, 4.5, 5, 6, 7, 8, 9 or 10 or more indicates that the immune checkpoint inhibitor or a combination of two or more thereof or a combination of an immune checkpoint inhibitor and other antitumor drugs is effective in the treatment of the cancer of interest.

14. A bispecific T-cell engage antibody for use in a method for predicting clinical response of a cancer patient to treatment by an immune checkpoint inhibitor or a combination of two or more thereof or a combination of an immune checkpoint inhibitor and other antitumor drugs, wherein
1) the bispecific T-cell engage antibody is contacted with a co-culture system comprising primary tumor cells from the cancer patient and lymphocytes containing T cells in the presence or absence of the immune checkpoint inhibitor or a combination of two or more thereof or a combination of an immune checkpoint inhibitor and other antitumor drugs; and
2) the release of IFN-γ due to the activation of T cells by the bispecific T-cell engage antibody is measured to determine whether the release of IFN-γ is increased in the presence of the immune checkpoint inhibitor or a combination of two or more thereof or a combination of an immune checkpoint inhibitor and other antitumor drugs as compared to the one absence thereof.

15. A bispecific T-cell engage antibody for use in a method for screening an immune checkpoint inhibitor or a combination of two or more thereof or a combination of an immune checkpoint inhibitor and other antitumor drugs effective in the treatment of a cancer of interest, wherein
1) the bispecific T-cell engage antibody is contacted with a co-culture system comprising primary tumor cells from the cancer patient and lymphocytes containing T cells in the presence or absence of the immune checkpoint inhibitor or a combination of two or more thereof or a combination of an immune checkpoint inhibitor and other antitumor drugs; and
2) the release of IFN-γ due to the activation of T cells by the bispecific T-cell engage antibody is measured to determine whether the release of IFN-γ is increased in the presence of the immune checkpoint inhibitor or a combination of two or more thereof or a combination of an immune checkpoint inhibitor and other antitumor drugs as compared to the one absence thereof.

16. The method of any one of claims 1-13 and the bispecific T-cell engage antibody of claim 14 or 15, wherein the bispecific T-cell engage is a bispecific single chain antibody construct containing at least two domains, wherein one of the domains specifically binds to antigens selected from human CD3, CD4, or CD8 antigen, and the second domain specifically binds to human tumor antigens selected from EpCAM antigen, EGFR antigen, CEA antigen, Her2 antigen, CD19 or CD20.

17. The method of any one of claims 1-13 and the bispecific T-cell engage antibody of claim 14 or 15, wherein the bispecific T-cell engage is an anti-EpCAM×CD3 bispecific T-cell engage, e.g., comprising an amino sequence of SEQ NO 1.

18. The method of any one of claims 1-13 and the bispecific T-cell engage antibody of claim 14 or 15, wherein the cancer is a solid tumor or a liquid tumor.

19. The method of any one of claims 1-13 and the bispecific T-cell engage antibody of claim 14 or 15, wherein the cancer or the solid tumor is selected from gastric cancer, colon cancer, colorectal cancer, breast cancer, ovary cancer or a combination thereof.

20. The method of any one of claims 1-13 and the bispecific T-cell engage antibody of claim 14 or 15, wherein the immune checkpoint inhibitor is an antibody or a fragment antigen binding thereof, or a chemical molecule drug.

21. The method of any one of claims 1-13 and the bispecific T-cell engage antibody of claim 14 or 15, wherein the immune checkpoint inhibitor is a chemical molecule drug, which inhibits one or more checkpoint proteins selected from human PD-1, TIM-3, A2AR, B7-H3(CD276), B7-H4(VTCN1), BTLA, CTLA-4, IDO, KIR, LAG3, or VISTA.

22. The method of any one of claims 1-13 and the bispecific T-cell engage antibody of claim 14 or 15, wherein the immune checkpoint inhibitor is an antibody or a fragment antigen binding thereof, which specifically binds to one or more checkpoint proteins selected from human PD-1, TIM-3, A2AR, B7-H3 (CD276), B7-H4 (VTCN1), BTLA, CTLA-4, IDO, LAG3, or VISTA.

23. The method of any one of claims 1-13 and the bispecific T-cell engage antibody of claim 14 or 15, wherein the immune checkpoint inhibitor is an antibody or a fragment antigen binding thereof, which specifically binds to PD-1.

24. The method of any one of claims 1-13 and the bispecific T-cell engage antibody of claim 14 or 15, wherein the immune checkpoint inhibitor is selected from nivolumab, pembrolizumab, Mab-1 comprising an amino sequence of SEQ NO 2.

25. The method of any one of claims 1-13 and the bispecific T-cell engage antibody of claim 14 or 15, wherein the other anti-tumor drug may have various modes of actions selected from immune therapies, targeted therapies, chemotherapies, vaccines, radiotherapies or combination thereof.

26. The method of any one of claims 1-13 and the bispecific T-cell engage antibody of claim 14 or 15, wherein the other anti-tumor drug is selected from a chemical molecule drug, a proteasome inhibitor, a histone deacetylase inhibitor, a glucocorticoid, a steroid, a targeted therapy agent or combination thereof.

27. The method of any one of claims 1-13 and the bispecific T-cell engage antibody of claim 14 or 15, wherein the other anti-tumor drug is a B-raf inhibitor.

28. The method of any one of claims 1-13 and the bispecific T-cell engage antibody of claim 14 or 15, wherein the other anti-tumor drug is 5-(((1R,1aS,6bR)-1-(6-(trifluoromethyl)-1H-benzo[d]imidazol-2-yl)-1a,6b-dihydro-1H-cyclopropa[b]benzofuran-5-yl)oxy)-3,4-dihydro-1,8-naphthyridin-2(1H)-one or a pharmaceutically acceptable salt thereof.

29. The method of any one of claims 1-13 and the bispecific T-cell engage antibody of claim 14 or 15, wherein the other anti-tumor drug is 5-(((1R,1aS,6bR)-1-(6-(trifluoromethyl)-1H-benzo[d]imidazol-2-yl)-1a,6b-dihydro-1H-cyclopropa[b]benzofuran-5-yl)oxy)-3,4-dihydro-1,8-naphthyridin-2(1H)-one Sesqui-Maleate.

30. The method of any one of claim 1 or 2, wherein the primary tumor cells are derived from a tumor biopsy, or a tumor tissue sample of surgery.

31. The method of any one of claims 1-13 and the bispecific T-cell engage antibody of claim 14 or 15, wherein the primary tumor cells are cultured on feeder cells, or in Matrigel®.

32. The method of any one of claims 1-13 and the bispecific T-cell engage antibody of claim 14 or 15, wherein the primary tumor cells were re-suspended with a medium comprising a substance listed as follows, before culturing,
1) F12/DMEM 1:1 medium 500 mL;
2) Fetal Bovine Serum 50 mL;
3) Insulin-Transferrin-Selenium (ITS-G) 0.5×;
4) Penicillin-Streptomycin 1×;
5) HEPES 1×;
6) Glutamine 2 mM;
7) MEM Non-Essential Amino Acids Solution 1×; and
8) Sodium pyruvate 1×.

33. The method of any one of claims 1-13 and the bispecific T-cell engage antibody of claim 14 or 15, wherein the lymphocytes containing T cells are derived from peripheral blood.

34. The method of any one of claims 1-13 and the bispecific T-cell engage antibody of claim 14 or 15, wherein the lymphocytes containing T cells are derived from allogeneic PBMCs, or autologous PBMCs.

35. The method of any one of claims 1-13 and the bispecific T-cell engage antibody of claim 13 or 14, wherein the lymphocytes containing T cells are derived from tissue selected from allogeneic TILs, autologous TILs, allogeneic lymphocytes from lymph node, autologous lymphocytes, ascites from lymph node, allogeneic spleen, autologous spleen, allogeneic ascites, or autologous ascites.

36. The method of any one of claims 1-13 and the bispecific T-cell engage antibody of claim 14 or 15, wherein the lymphocytes containing T cells can be replaced by purified CD3+ cells or CD4+ cells.

37. The method of any one of claims 1-13 and the bispecific T-cell engage antibody of claim 14 or 15, wherein the PBMCs can be replaced by purified CD3+ cells or CD4+ cells.

38. The method of any one of claims 1-13 and the bispecific T-cell engage antibody of claim 14 or 5, wherein the PBMCs are pre-activated by an antibody specifically binding to human CD3, CD4, or CD8 antigen.

39. The method of any one of claims 1-13 and the bispecific T-cell engage antibody of claim 14 or 15, wherein the PBMCs are pre-activated by an anti-CD3 antibody.

40. The method of any one of claims 1-13 and the bispecific T-cell engage antibody of claim 14 or 15, wherein the control groups are groups further adding an isotype control antibody.

41. The method of any one of claims 1-13 and the bispecific T-cell engage antibody of claim 13 or 14, wherein the control groups are groups further adding an isotype control IgG.

42. The method of any one of claims 1-13 and the bispecific T-cell engage antibody of claim 14 or 15, wherein the tIFN-γ/cIFN-γ ratio is determined by simultaneously measuring tIFN-γ amount and cIFN-γ amount with flow cytometry, ELISPOT or ELISA.

43. The method of any one of claims 1-13 and the bispecific T-cell engage antibody of claim 14 or 15, wherein the tIFN-γ/cIFN-γ ratio is determined by simultaneously measuring tIFN-γ amount and cIFN-γ amount with ELISA.
